(19) United States
(12) Patent Application Publication
(10) Pub. No.: US 2010/0042029 A1
(43) Pub. Date: Feb. 18, 2010

(54) OSTEOARTHRITIS KNEE ORTHOSIS

(76) Inventor: Richard A. Nace, San Jose (CR)

Correspondence Address: James E. Larson, Esq., 1717 North Bayshore Drive, Suite 240, Miami, FL, 33132, US

(21) Appl. No.: 12/606,166

(22) Filed: Oct. 26, 2009

Related U.S. Application Data
(63) Continuation-in-part of application No. 11/556,557, filed on Nov. 3, 2006, now Pat. No. 7,608,051.

(51) Int. Cl. A61F 5/00

(52) U.S. Cl. 602/16; 602/26

(57) ABSTRACT

A polycentric hinge is positioned on one side of the patient's knee and a unicentric hinge is positioned on a second side of the knee. A lower portion of the polycentric and unicentric hinge is attached to a knee cuff about a lower leg of the patient. An upper arm of the polycentric and unicentric hinge is attached to a thigh cuff, the upper arm of the unicentric hinge being longer than the upper arm of the polycentric hinge. The upper arm of the unicentric hinge has a 15° to 20° set back angle when the patient's leg is straight. The polycentric hinge has a slotted central plate with star gears engaging a back portion and multiple movable blocks mounted in screw holes on a front edge of the slotted plate supporting an elastic band having a fulcrum point on the polycentric hinge at a lower and upper end.

### PRIOR APPLICATIONS

This application is a continuation-in-part patent application of U.S. Ser. No. 11/556,557, filed Nov. 3, 2006, currently allowed and scheduled to issue on Oct. 27, 2009 as U.S. Pat. No. 7,608,051.

### FIELD OF THE INVENTION

The invention relates to knee braces. More particularly, it relates to a knee brace used to treat osteoarthritis of the knee, knee joint injury and other neurological knee joint disease, which rehabilitates abnormal gait due to said above mentioned conditions.

### BACKGROUND OF THE INVENTION

Orthotic devices and appliances commonly referred to as “orthotics,” have been utilized for many years by orthotists, physical therapists, and occupational therapists to assist in the rehabilitation of patient's joints and associated limbs or adjacent skeletal parts of the patient's body.

Webster's New College Dictionary defines “orthotics” as a branch of mechanical and medical science that deals with the support and bracing of weak or ineffective joints or muscles.

Orthotics or limb braces have been designed to support and protect the joint, alleviate pain associated with joint movement, and to rehabilitate the joint over time with orthotic use.

Primary osteoarthritis is usually related to aging. With aging, the water content of the cartilage increases and the protein makeup of the cartilage degenerates. Repetitive use of the joints over the years can irritate and inflame the cartilage, causing joint pain and swelling. Eventually, cartilage begins to degenerate by flaking or forming tiny crevasses. In advanced cases, there is a total loss of cartilage cushion between the femur and tibia bones at the knee joint, leading to diminished joint space on the affected side of the knee resulting in pain and limitation of joint mobility. Inflammation of the cartilage also can stimulate new bone outgrowths (spurs) to form around the joints causing increased pain and joint inflammation.

Osteoarthritis is often described as “wear and tear” arthritis, as it is highly correlated to age. Osteoarthritis is one of the most frequent causes of physical disability among adults. More than 20 million people in the US have the disease. By 2030, 20 percent of Americans, about 70 million people, will have passed their 65^{th} birthday and will be at risk for osteoarthritis.

Osteoarthrosis is a condition where the joint is affected by degeneration. Osteoarthritis implies the same meaning, but the “itis” adds the meaning that the joint is inflamed. The two terms are often used interchangeably.

Joint replacement surgery of the knee is the surgical treatment for osteoarthrosis or osteoarthritis. It is best to delay knee joint replacement surgery as long as possible, as a total knee replacement may need to be replaced in ten to twenty years. It is a major surgery which requires considerable rehabilitation therapy to restore full function.

Exercise, weight loss if needed, and the use of anti-inflammatory medications and analgesics are often prescribed to assist the patient in managing the pain associated with osteoarthritis. Minimizing the progression of the damage to the cartilage of the knee joint and preventing the formation of bone spurs from “bone on bone” during knee joint bending is an important part of patient care.

The actual pain of osteoarthritis or osteoarthrosis comes from wearing away of the soft cartilage that pads the junction of the femur (upper leg bone of the knee) and the tibia (lower leg bone of the knee). With irritation of the joint, bone spurs can form causing bits of bone and cartilage to break off which float inside the joint space further irritating the knee. The most common form of osteoarthritis or osteoarthrosis is unicompartmental, meaning that only one of the three compartments of the knee joint are significantly affected by the loss of cartilage padding. The medial compartment of the knee is on the inside of the center line of the body. The lateral compartment of the knee is on the outside plane of the body, and the patellar compartment is in the center top of the knee behind the patella or knee cap. The majority of cases of osteoarthritis are medial compartment degeneration where the cartilage or cushioning of the knee joint has significantly deteriorated. The knee then becomes imbalanced, with the knee bowing outwards. This is often called a “bowleg” condition. A “bowleg” (genu varum), commonly referred to as a valgus deformity of the knee joint, places significant force on the medial compartment of the knee, which aggravates the pain associated with osteoarthritis when the patient walks, bends the knee, or stands up.

As the cartilage or padding of the knee joint on the lateral compartment cartilage is worn away, the knee will deform abnormally bending inwards at the knee joint giving the patient a knock kneed appearance. This is referred to as a varus deformity of the knee joint.

Osteoarthritis knee braces are designed to do two things: first, correct the abnormal bending of the knee joint inwards or outwards (varus or valgus correction). Secondly, many osteoarthritis knee orthotics or braces are designed to prevent the “bone on bone” contact of the femur and tibia bones in the medial or lateral compartment of the knee joint as the patient bears weight during ambulation. This action of lifting femur, pulling down the tibia or keeping the femur and tibia bones from coming in contact during the straightening of the knee during heel strike is often called “unloading” the knee joint. By “unloading” the knee joint, the constant irritation of the degenerated cartilage in the affected compartment of the knee (medial or lateral) can lead to a significant reduction in pain and further injury to the knee joint. Osteoarthritis knee braces also provide improved alignment of the upper and lower aspects of the knee joint by preventing the bending inwards or outwards of the knee joint during gait. These two features, unloading and alignment are provided by most of the osteoarthritis knee orthotics available in today's market.

The majority of knee orthotics available to treat osteoarthritis of the knee utilizes a single upright attached to an upper thigh cuff and lower shin cuff. The upright is located on the side of the collapsed compartment of the knee; i.e. medial side for medial compartment osteoarthritis. The attached cuffs “offload” the biomechanical force on the affected compartment of the knee by increasing the joint space on the affected side as the knee goes from flexion to extension. Many osteoarthritis braces use an angled strap from the upper part of the brace that goes across the opposite side of the knee joint from the side bar or upright to improve the alignment of the knee during ambulation to better balance the forces on the knee during gait more evenly. The strap provides a three point leverage that pulls the knee joint into proper alignment during gait. A combination of the single sided upright with cuff attachments and the valgus producing strap have shown to provide improved performance in severe genu varum osteoarthritis. However, it is difficult to set the desired degrees of flexion and extension.

Although many of the existing knee braces containing locking hinge assemblies serve their intended purpose, difficulty in ease of setting the desired degrees of flexion and extension continues to be a problem.

### SUMMARY OF THE INVENTION

The present invention provides an osteoarthritis knee orthosis easily fabricated in a wide range of sizes for either knee (left or right) to treat either medial or lateral (varus or valgus) unicompartmental degeneration of the knee joint caused by osteoarthritis or osteoarthrosis with easily managed controls for setting the desired degree of flexion and extension. The knee orthosis of this invention will first unload the pressure on the affected side of the knee joint and thereby provide balanced joint space on both sides of the knee during ambulation. This then improves knee joint alignment. Adjustments to the knee orthosis can be made as the condition of the patient's knee improves or deteriorates to maintain a proper joint space balance, sustain an unloading effect on the effected side of the knee joint, and for improving knee joint alignment during gait. The knee orthosis can employ either a single upright member or two generally parallel upright members positioned opposed from one another on either side of an affected knee joint of a patient's leg.

Through the use of the present invention, as alignment of the knee changes (movement towards optimal or at least significantly improved joint space balance), the mechanism of the knee brace can be adjusted so that joint space balance is continually maintained with joint rehabilitation. The current invention achieves this significant improvement by employing a gait swing assist mechanism, which in the preferred embodiment is an adjustable dynamic fulcrum. The gait swing assist mechanism allows the clinician to quickly and easily adjust the brace to maintain joint space balance as needed during the knee rehabilitation process. The gait swing assist mechanism of the present invention also assists in returning abnormal gait that may include improper heel-to-toe placement to a more normal gait. In such a case, when returning to a more normal gait, all of the muscles of the upper and lower leg and those at the knee joint area are used and a corrective muscle firing sequence occurs, thereby preventing muscle atrophy and reactivating the muscles. This then results in muscle rehabilitation, muscle strengthen and muscle growth, after injury or disease, wherein the gait had be altered (i.e., the gait had become abnormal). The ultimate goal, and one which is achieved with the orthosis of the present invention, is the facilitation of normal gait mechanics (i.e., proper heel-to-toe placement) through the corrective actions of the orthosis and a reactivation of leg muscle memory, which is defined as the precise sequence or order in which the muscles contract and/or elongate. It should also be noted that when speaking of muscle strengthening and muscle growth, what is meant is the muscles above and below the knee as well those muscles at and around the knee joint area.

The present invention first accomplishes the desired result of knee joint space balance by providing, in the preferred embodiment, a polycentric hinge and a medial unicentric hinge component, one positioned laterally while the other is positioned medially adjacent the knee joint, depending on which side of the knee joint is affected by osteoarthritis. However, nothing herein, in alternate embodiments of the present invention, limits the use of two unicentric hinges or two polycentric hinges or for that matter just one upright with just one hinge that is either a unicentric or polycentric hinge.

A shin cuff is circumscribable about the lower leg below the knee joint (i.e., shin and calf) either posteriorly or anteriorly. A lower member of each polycentric and unicentric hinge, in the preferred embodiment, is attached to an upright element integral with the shin cuff. A thigh cuff is circumscribable about the upper leg either posteriorly or anteriorly with the patient's thigh, above the knee joint. The thigh cuff has opposed lateral and medial elements extending downwardly to engage a top hinge arm of the lateral and medial hinge, respectively. In an offset design of the preferred embodiment, the top medial hinge arm is longer than a corresponding top lateral hinge arm. The top medial hinge arm is also fixed at a posterior angle of about 15° to 20° in full knee extension, although lesser or larger angles can be employed in alternate embodiments. Further, nothing herein limits that the osteoarthritis knee orthosis of the present invention be offset at all. Still further, the thigh and shin cuffs can both be either rigid or semi-rigid or any combination of both rigid and semi-rigid. Still even further, any orientation for the thigh and shin cuffs can be employed and include, but is not limited to, both the thigh and shin cuff being posteriorly oriented, both the thigh and shin cuff being anteriorly oriented, or the thigh cuff being posteriorly oriented, while the shin cuff is anteriorly oriented, or the thigh cuff being anteriorly oriented, while the shin cuff is posteriorly oriented.

A spring loaded dynamic adjustable fulcrum is used to assist in leg extension-flexion during walking, squatting and sitting. By moving a plurality of setting blocks or varying tension springs, different degrees of tension can be introduced by the dynamic adjustable fulcrum. In the preferred embodiment, the dynamic adjustable fulcrum is located on an outer side of the polycentric hinge. However, nothing herein limits use thereon and therefore in alternate embodiments, the dynamic adjustable fulcrum is located along the outer side of a unicentric hinge.

### BRIEF DESCRIPTION OF THE DRAWINGS

This invention can be best understood by those having ordinary skill in the art by reference to the following detailed description, when considered in conjunction with the accompanying drawings in which:

FIG. 1 is a lateral side perspective view of the hinge assembly of this invention including the attachment strapping.

FIG. 2 is a medial side perspective view of the hinge assembly of FIG. 1.

FIG. 3 is a front view of a knee with a hidden view of the patella out of alignment.

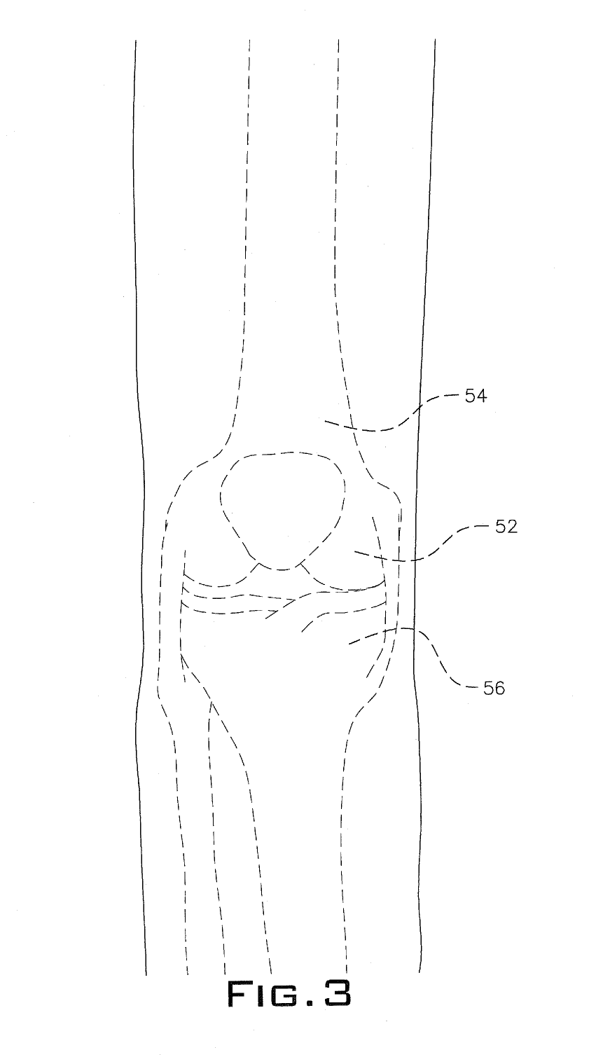

FIG. 4 is a front view of a knee with a hidden view of the patella with the knee brace of this invention in place.

FIG. 5A is a lateral side view of the hinge assembly of FIG. 1 on an extended leg of a patient.

FIG. 5B is a lateral side view of the hinge assembly of FIG. 5A with the patient's knee in a bent position.

FIG. 6A is a lateral side view of the hinge assembly of FIG. 1 on an extended leg of a patient with only two setting blocks in place.

FIG. 6B is a lateral side elevational view according to FIG. 6A with the patient's knee bent.

FIG. 7A is a lateral side view of the hinge assembly of FIG. 1 on an extended leg of a patient with only one setting block in place.

FIG. 7B is a lateral side according to FIG. 7A with the patient's knee bent.

FIG. 8A is a lateral side view according to FIG. 1 on an extended leg of a patient with the setting blocks moved to an extreme position.

FIG. 8B is a lateral side view according to FIG. 8A with the patient's knee bent.

### DETAILED DESCRIPTION OF THE INVENTION

Throughout the following detailed description the same reference numerals refer to the same elements in all figures.

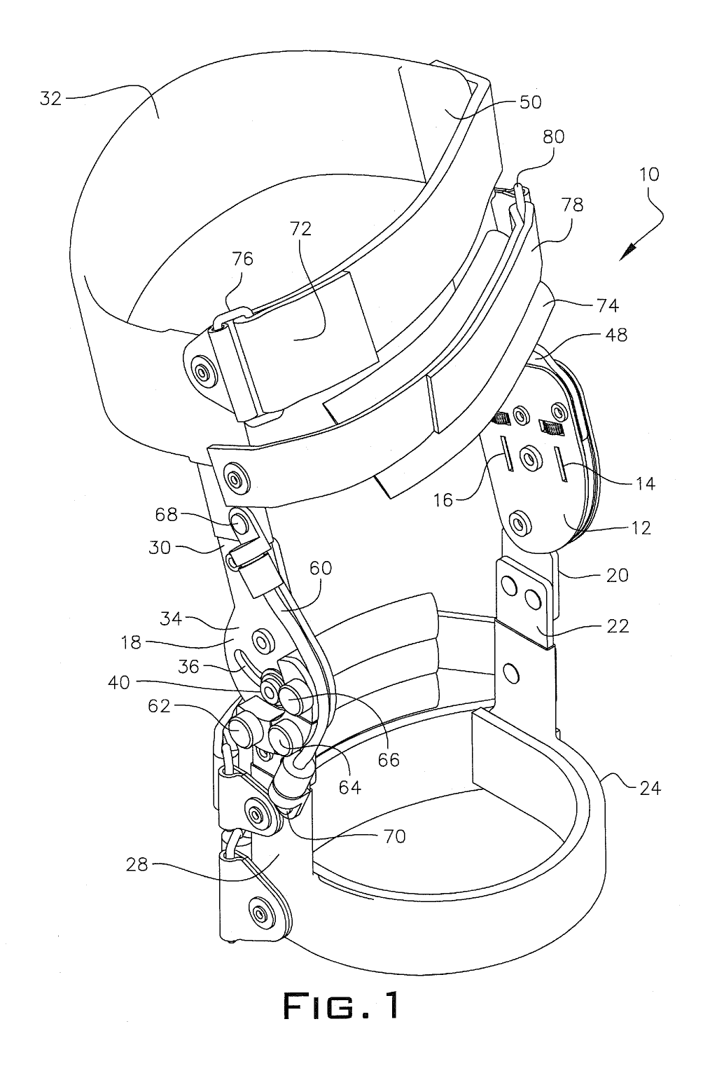

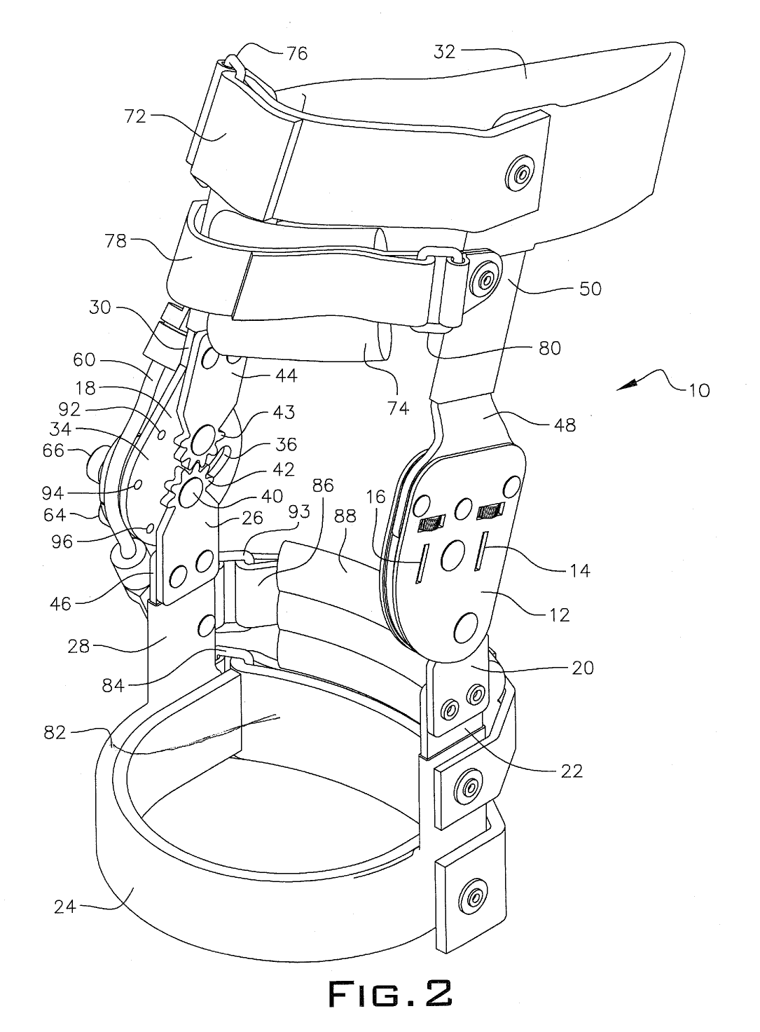

Referring to FIGS. 1 and 2, an offset osteoarthritis knee orthosis 10 (hereafter identified as OA) of the present invention in a preferred embodiment is shown, wherein a medial unicentric hinge 12 has a flexion stop 14 and extension stop 16 at any setting. Alternate embodiments (not shown) are not offset. The medial unicentric hinge 12 is substantially parallel to a lateral polycentric adjustable tension offloading hinge 18. The bottom end 20 of medial hinge 12 is attached to a first upright member 22 integral with a knee ring 24. A lower gear plate 26 of lateral hinge 18 is attached to a second upright member 28 integral with knee ring 24.

An upper arm 30 of the lateral hinge 18 connects at an upper end to an upper thigh cuff 32. A lower portion of the lateral hinge broadens out to a slotted hinge connector plate 34. A slot 36, in connector plate 34 contains a transverse shaft on rivet 40. The rivet 40 attaches a first star gear 42 to an inside surface of the slotted connector plate 34. A second star gear 43 is integral with a gear plate 44 attached to an inner surface of upper arm 30. In alternate embodiments of the present invention (not shown) other polycentric hinge mechanisms are employed that do not require the use of the star gears.

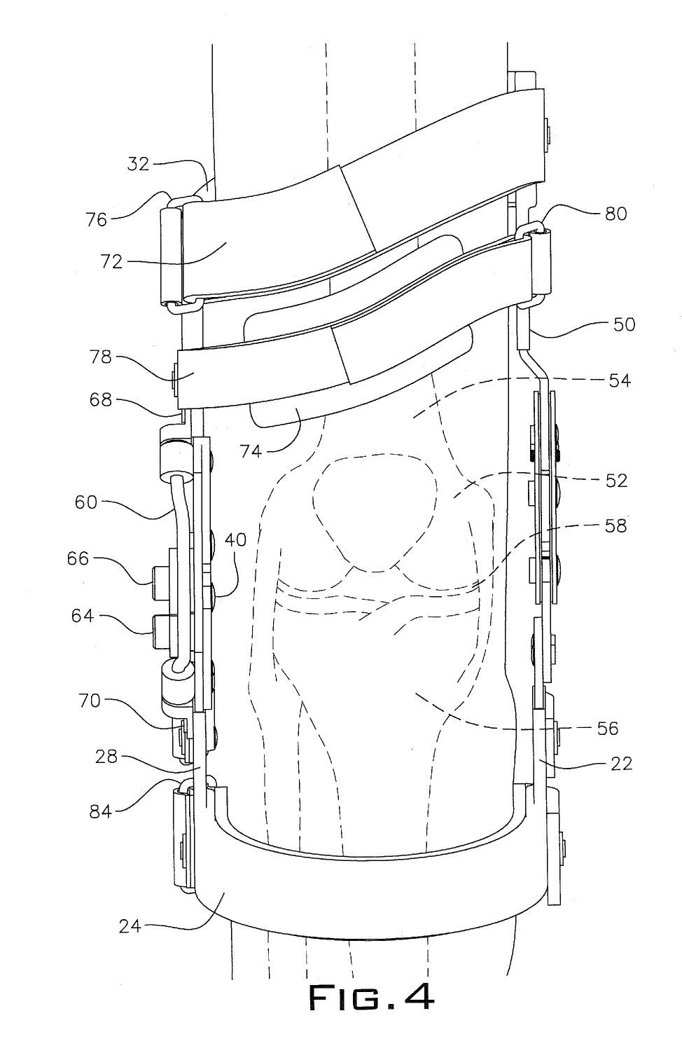

An upper arm 48 of the medial hinge 12 is attached at its top end 50 to the upper thigh cuff 32. In the preferred embodiment, medial hinge 12 is a KWIK-SET™ unicentric design as set forth in U.S. Pat. No. 6,039,709, which is accordingly incorporated herein by reference. However, it should be noted that alternate embodiments of the present invention employ other types of unicentric hinges that function differently than the preferred KWIK-SET™ as shown herein. The upper arm 48 of medial hinge 12 is longer than the upper arm 30 of the lateral hinge set 18; about 1 and 2 inches in the medium size. The longer length of medial upper arm 48 compared to the upper arm 30 of the lateral hinge 18 encourages the unloading of force from the medial compartment of the knee by lifting the medial side 52 of the femur 54 off of the tibia 56 with weight bearing during full leg extension. See the gap 58 shown in FIG. 4. It should be noted though that nothing herein limits the use of the longer medial hinge upper arm. In fact, in one alternate embodiment, the lateral hinge upper arm is longer than the medial hinge upper arm and in another alternate embodiment the medial and lateral upper arms are of equal length.

The lower medial side upright 22 and lower lateral side upright 28 are equivalent in length. In the preferred offset embodiment, the medial upper arm 48 is set back at an angle of approximately 15° to 20° with the leg straight whereas upper lateral arm 30 and lower lateral upright 28 are in a relatively straight alignment with the hinge when the leg is straight. The offset portion of arm 48 improves knee alignment from 20° of flexion to full extension of the knee joint and prevents “reverse Screw Home Mechanism” rotation of the knee. Other degrees and ranges of angles can be employed for the offset, when employed, and nothing herein limits the use of the ranges as set forth above.

As shown, the various elements are held together by rivets such as rivet 40. However, other equivalent means and elements of attachment can be substituted for the rivets and therefore nothing herein limits the present invention to the use of just rivets. Other examples of alternate attachment means and elements, which are not meant to be exclusive, include buckles, hooks, D-rings and other similar shaped-ring attachments, friction snap elements, screw mechanisms, hooks, glue, stitching and knotted arrangements.

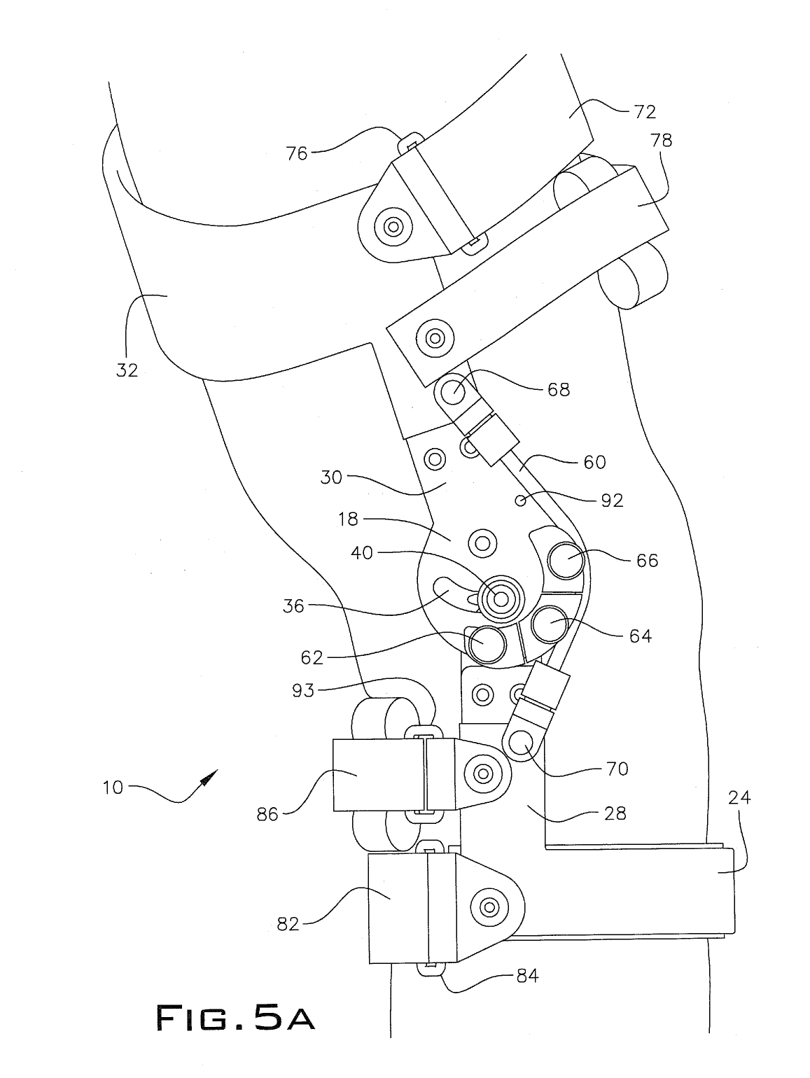

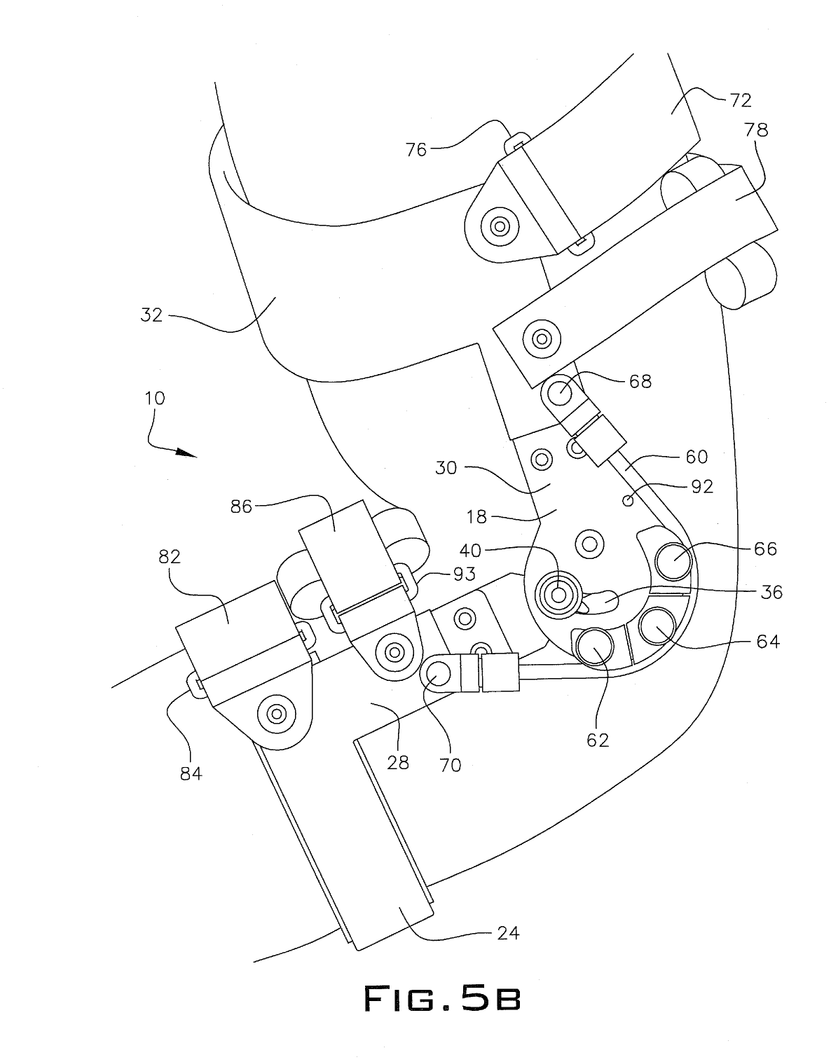

On polycentric hinge 18, an elastic band 60 attaches at a lower end on the outside of the second upright member 28 at fulcrum point 70 and travels adjacent adjustable dynamic fulcrum setting blocks 62, 64 and 66 to terminate at fulcrum 68. The dynamic tension of the fulcrum 68 can be set by the fitter by using fulcrum blocks 62, 64 and 66. In addition, various elastic bands 60 with varying elastic properties can be substituted to allow the fitter to adjust the dynamic fulcrum with multiple tension forces. The dynamic adjustable fulcrum is derived from the band 60 positioned from the lower fulcrum point 70 and stretched over blocks 62, 64 and 66 at the lateral hinge as the knee bends (see FIG. 5B). The adjustable dynamic fulcrum is used to provide a dynamic tension force at the knee joint that can be used to balance the joint space 58 between the medial and lateral compartments and to provide optimal alignment of the knee with the OA 10 brace as well as assist in correcting abnormal gait such as facilitating a more normal heel-to-toe placement. Further, the dynamic adjustable fulcrum is just one example of the gait swing assist mechanism that can be used with the present invention. Other mechanisms can be employed and are contemplated herein, such as an internal hinge spring mechanism with no fulcrum. However, if the dynamic adjustable fulcrum is employed, it can be either a single or variable fulcrum and can employ either the elastic band, as described above, or another similar acting element, like a spring stretched over the blocks.

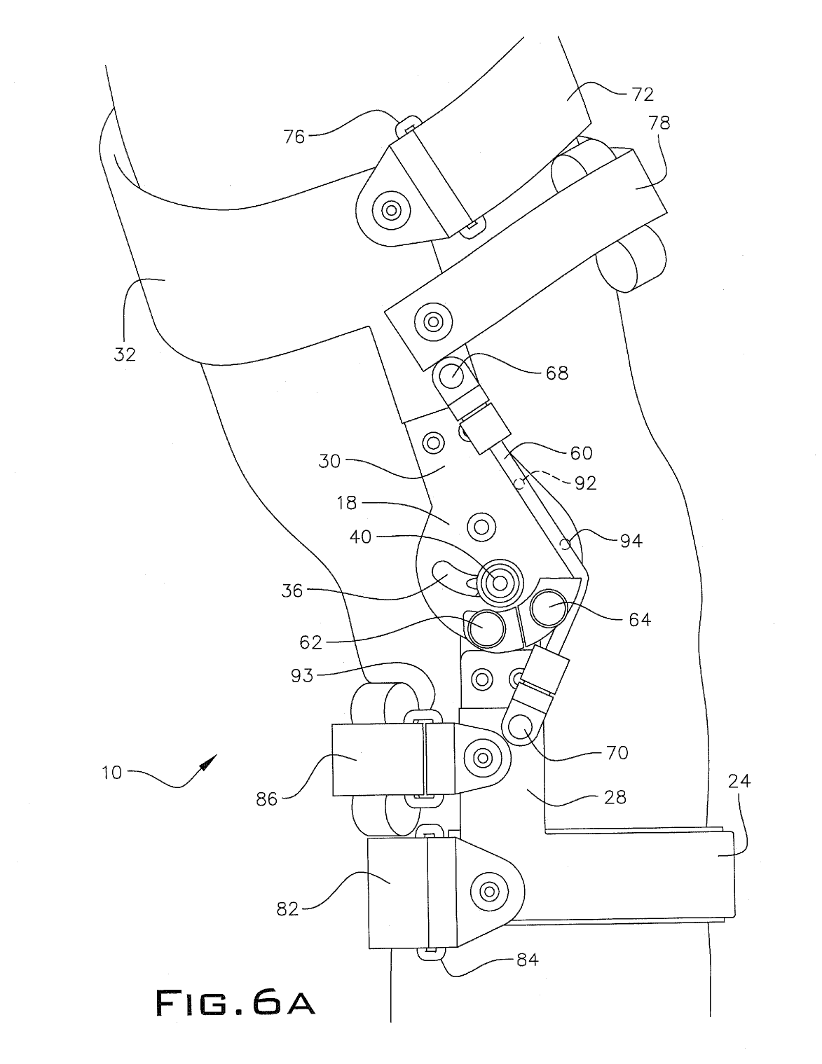

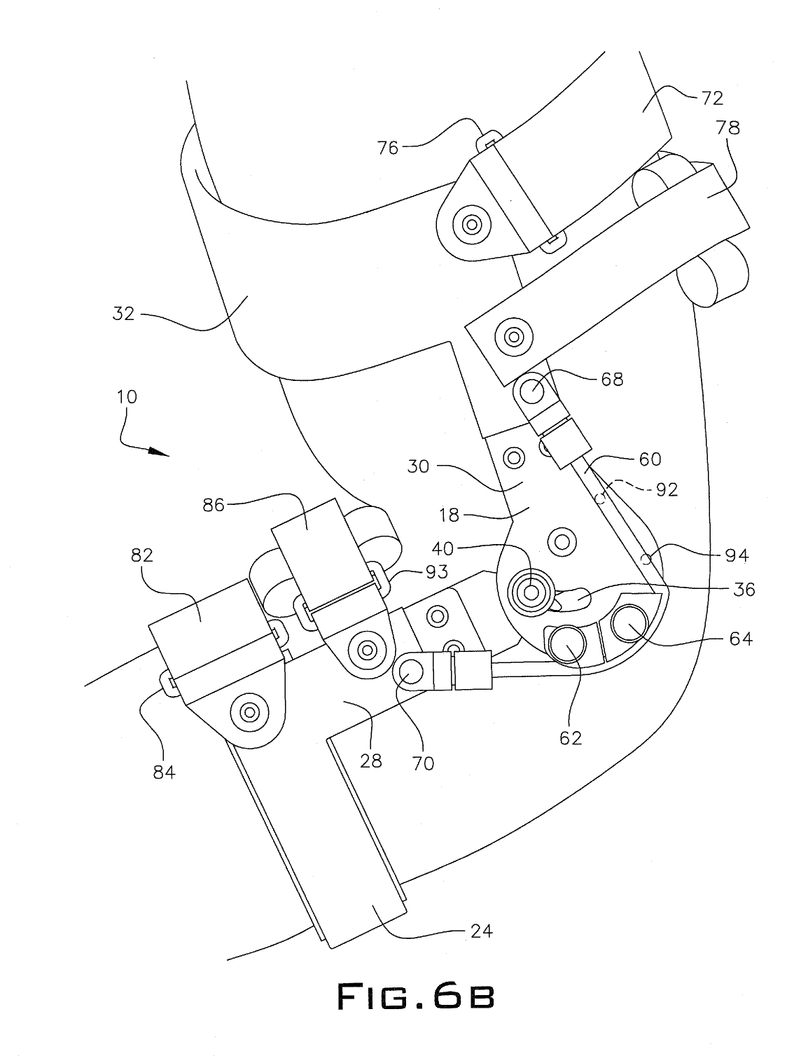

FIGS. 6A and 6B show alignment using only two blocks, 62 and 64, on the polycentric hinge 18.

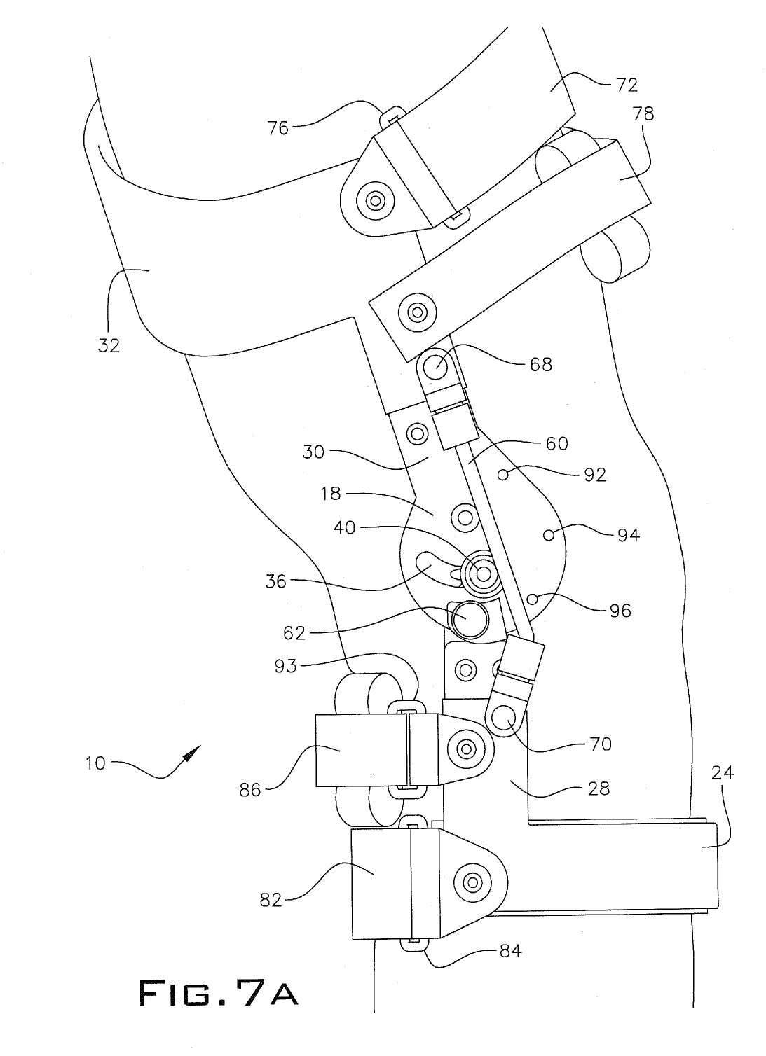

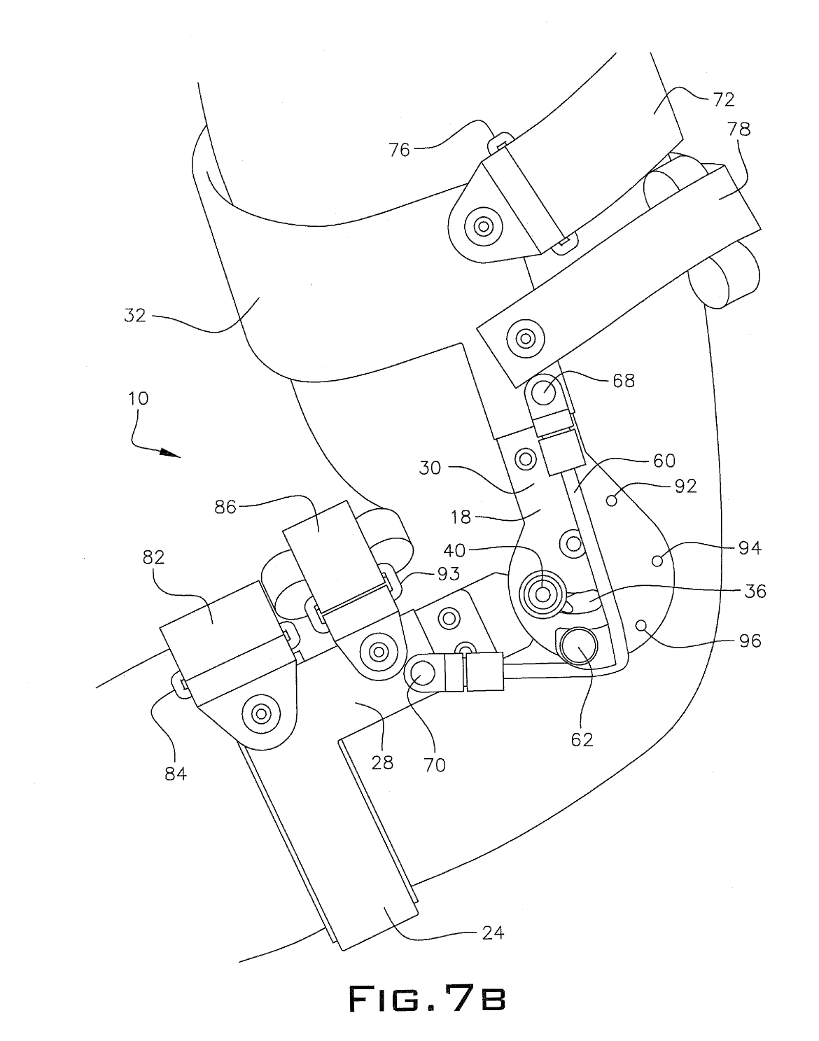

FIGS. 7A and 7B show minimal alignment using only one block 62 on the polycentric hinge 18.

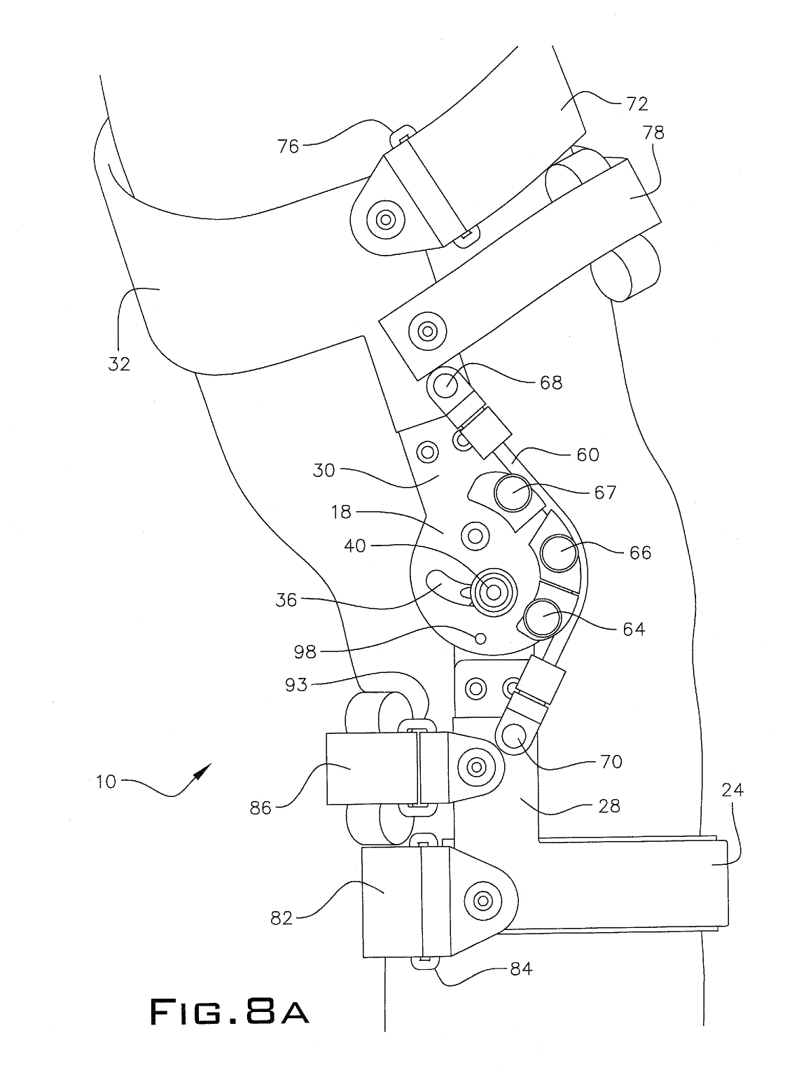

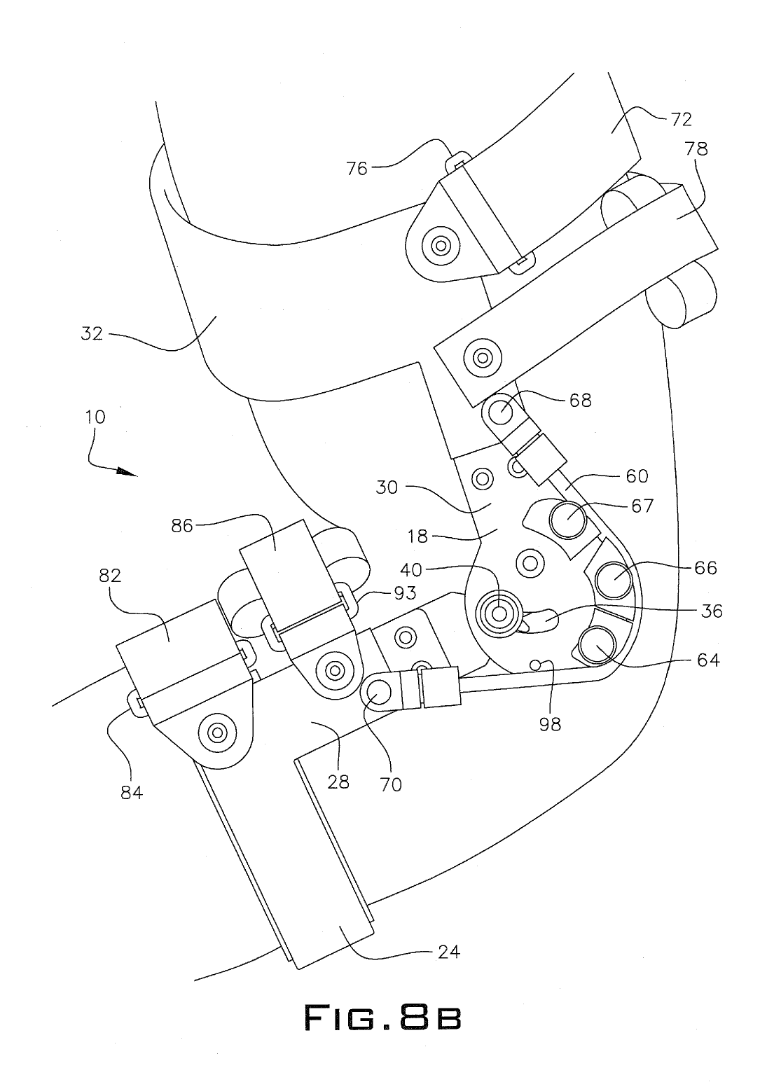

FIGS. 8A and 8B show an extreme alignment using blocks 64, 66 and 67. In this condition there is no block 62 in lower aperture 98 on the polycentric hinge 18.

A strap 72 tightens thigh cuff 32 in place.

A second mid-thigh cuff securing strap 78 is a soft elastic material with a soft adjustable pad 74 placed at the inner midpoint area of strap 78. Strap 72 is placed through a D-ring fastener 76 on the side of the thigh cuff 72. Hook and loop material is used to engage strap 72 to itself. Soft padded mid-thigh securing strap 78 travels from the lateral side of OA 10 through a second D-ring fastener 80 on the medial side of OA 10. The strap 78 fastens to itself by hook and loop material.

A shin cuff soft elastic material securing strap 82 travels across the back of the patient's calf through a third D-ring fastener 84 on the lower end of the lateral upright 28 and fastens onto itself with hook and loop material. A padded calf strap 86 travels around the back of the calf with an adjustable pad 88 located at mid strap 86. Strap 86 passes through a D-ring fastener 93 on the lower upright 28 and is attached to itself by hook and loop material. As stated before with the use of rivets 40, nothing herein limits the use of D-ring fasteners, as described directly above, and it should therefore be understood that other alternate attachment means and elements can be employed and include, but are not limited to, buckles, hooks, non D-shaped ring attachments, friction snap elements, screw mechanisms, hooks, glue, stitching and knotted arrangements.

The knee brace 10 bends with free moving axial hinges, each with six equivalently sized axial teeth on the upper and lower aspects of the inner hinge assembly of the medial and lateral hinges 12 and 18. As the knee bends, the bend movement is tracked or guided by the rigid brace uprights at the hinge center axle through groove 36 on the lateral hinge. The hinge alignment forces the bend line of the brace through grooves 36 that is pre-cut into the lateral hinge 18. The specific bend movement of the knee is controlled by the hinge axle traveling through the grooves 36 in the lateral hinge 18. The adjustable dynamic fulcrum on the lateral hinge 18 can be set to provide a dynamic assist mechanism 62, 64 and 66 with spring band 60 to assist in controlling the alignment and movement of the knee from 20° of flexion to full extension of the knee. The adjustable properties of the dynamic fulcrum offer the fitter or wearer multiple settings to maintain knee joint space balance, improved knee joint alignment, and prevention of “reverse Screw Home Mechanism” or controlled rotation of the knee as the knee goes from 20° flexion to extension.

The adjustable dynamic fulcrum band mechanism 60 is on the lateral upright of the OA Knee Brace 10 for medial compartment osteoarthritis. Four threaded holes 92, 94, 96 and 98 are arranged along the hinge of the outer lateral connector plate 34. Blocks 62, 64, 66 and 67 are screwed into the holes as needed. Block 64 can be inserted in hole 96 providing the center point of the elastic band fulcrum as the knee bends if the optimal dynamic setting of the adjustable pull mechanism is needed. A second block 62 with elastic band 60 tracking material can be inserted into threaded hole 98 if another optimal dynamic setting of the adjustable pull mechanism is needed. A third adjustable dynamic fulcrum mechanism is available by inserting block 66 with the elastic band 60 tracking material into threaded hole 94. By providing three or more incrementally stronger or more elastic bands, the fitter will have multiple settings to adjust the dynamic fulcrum to maintain the correct amount of dynamic force to maintain optimal joint space on both the medial and lateral compartments of the knee as well as to maintain optimal knee alignment during the motion of the knee during ambulation.

Other equivalent elements can be substituted for the elements disclosed herein to produce the same results in the same way.

Having thus described the invention, what is claimed for Letters Patent follows:

1. A knee brace for treating osteoarthritis of a patient's knee, the knee brace comprising:
a) at least one upright member attached to a shin cuff at a lower end and to a thigh cuff at an upper end, the shin cuff adapted to be circumscribable about a patient's lower leg below the knee and the thigh cuff adapted to be circumscribable about a patient's thigh above the knee;
b) at least one hinge element attached along the at least one upright member such that the at least one hinge element is positioned juxtaposed to a side of the knee when the knee brace is employed on the patient;
c) a gait swing assist mechanism mounted along an outer side of the at least one hinge element; and
d) a plurality of straps for securing the shin and thigh cuff to the patient's lower and upper leg portion below and above the knee respectively.

2. The knee brace of claim 1, wherein the at least one upright member comprises two upright members, a first upright member disposed along a lateral side of the patient's leg and a second upright member disposed along a medial side of the patient's leg.

3. The knee brace of claim 2, wherein the at least one hinge element comprises two hinge elements.

4. The knee brace of claim 3, wherein the two hinge elements are chosen from the group consisting of a unicentric hinge and a polycentric hinge.

5. The knee brace of claim 4, wherein the gait swing assist mechanism is mounted along an outer side of the polycentric hinge.

6. The knee brace of claim 1, wherein the gait swing assist mechanism comprises a dynamic adjustable fulcrum having at least one removable setting block and an elastic band stretched over said at least one removable setting block.

7. The knee brace of claim 1, wherein the gait swing assist mechanism comprises a dynamic adjustable fulcrum having a plurality of varying tension springs.

8. The knee brace of claim 4, wherein the hinges are chosen from the group combination of a polycentric hinge on the lateral side and a unicentric hinge on the medial side, a polycentric hinge on the medial side and a unicentric hinge on the lateral side, a polycentric hinge on both the lateral side and medial sides and a unicentric hinge on both the lateral side and medial sides.

9. The knee brace of claim 1, wherein the shin cuff is positioned on an anterior side of the patient's lower leg and the thigh cuff is positioned on a posterior side of the patient's upper leg.

10. The knee brace of claim 1, wherein the shin cuff is positioned on a posterior side of the patient's lower leg and the thigh cuff is positioned on an anterior side of the patient's upper leg.

11. The knee brace of claim 1, wherein the shin and thigh cuffs are either both positioned on the anterior side of the patient's leg or both positioned on the posterior side of the patient's leg.

12. The knee brace of claim 1, wherein the shin cuff is rigid and the thigh cuff is semi-rigid.

13. The knee brace of claim 3, wherein the first upright member is offset.

14. The knee brace of claim 13, wherein the offset first upright member has an upper arm of a hinge element that is longer than an upper arm of a hinge element of the second upright member.

15. The knee brace of claim 14, wherein the first upright member is offset such that the thigh cuff has one side positioned at a height higher than its opposed side, wherein the thigh and shin cuffs have different horizontal axis and wherein the upper arm of the first upright member
is angled backwardly from a vertical axis of the second upright member.

16. The knee brace of claim 4, wherein the unicentric hinge includes an extension and a flexion setting on a central plate of said hinge.

17. The knee brace of claim 1, wherein the thigh cuff attaches to a thigh cuff securing strap at opposed ends thereof by attachment devices for securing the knee brace around the patient's thigh.

18. The knee brace of claim 1, wherein the shin cuff attaches to a shin cuff securing strap at opposed ends thereof by attachment devices for securing the knee brace around a patient's shin and calf.

19. The knee brace of claim 6, wherein the at least one removable setting block comprises one to four removable setting blocks.

20. The knee brace of claim 19, wherein one removable setting block is employed in a lowermost position of the dynamic adjustable fulcrum.
